(19) United States
(12) Patent Application Publication
(10) Pub. No.: US 2010/0069684 A1
(43) Pub. Date: Mar. 18, 2010

(54) ASSYMMETRIC HYDROGENERATION OF PROCHIRAL COMPOUNDS

(76) Inventors: Johannes Gerardus De Vries, Maastricht (NL); Bart Stegink, Groningen (NL)

Correspondence Address: NIXON & VANDERHYE, PC, 901 NORTH GLEBE ROAD, 11TH FLOOR, ARLINGTON, VA, 22203, US

(21) Appl. No.: 12/520,681

(22) PCT Filed: Dec. 21, 2007
(86) PCT No.: PCT/EP07/11347

§ 371 Date: Nov. 6, 2009

(30) Foreign Application Priority Data
Dec. 22, 2006 (EP) 06026724.2

(51) Int. Cl. C07C 29/143

(52) U.S. Cl. 568/814

(57) ABSTRACT

The invention relates to the asymmetric (transfer) hydrogenation of a prochiral keton, prochiral imine, oxime, oxime derivative, hydrazone or hydrazone derivative, using a transition metal catalyst, characterized in that as a catalyst is used a transition metal complex of the general formula [I]: MₐL¹_{b}L²_{c}(N)_{d}Xₑ wherein a, b, c, d and e are integers; a, b and d can have a value of 1-6; c and e can have a value of 0-6; M is transition metal selected from the group consisting of Ru1 Rh and Ir; L¹ is an enantiomerically enriched chiral monodentate phosphor-containing ligand of the general formula [II] or of the general formula [III]; L² is any monodentate or bidentate neutral or monoanionic ligand, which may be chiral; N is a compound containing at least one primary or secondary amine group; X is a anion.

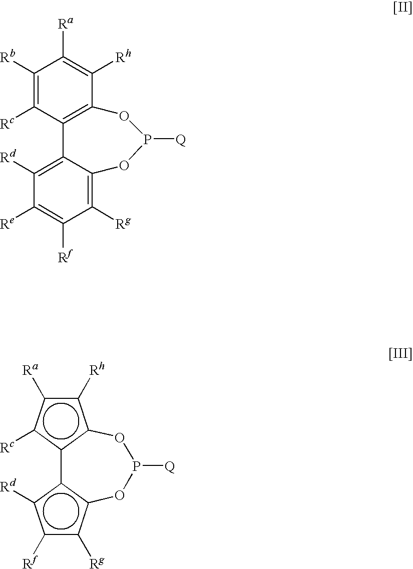

The present invention relates to a method for the production of enantiomerically enriched chiral alcohols and amines by asymmetric hydrogenation or asymmetric transfer hydrogenation of prochiral ketones, imines, oximes, oxime derivatives, hydrazones or hydrazone derivatives using a transition metal catalyst comprising an enantiomerically enriched chiral monodentate ligand.

Asymmetric hydrogenation of prochiral ketones has been described in EP 1325013 A1. In Example V of said patent application, the hydrogenation of acetophenone has been described with a Ruthenium catalyst comprising 1,2-diphenylethylene diamine as a ligand and comprising a ligand having to the following

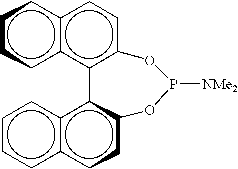

and a ligand wherein two N-methylgroups are replaced by i-propylgroup. However, the e.e. of that combination of ligands results in e.e's of 58% and 67%, respectively, only.

Asymmetric hydrogenation of prochiral ketones has been described by Junge et al. (Angew. Chem. Ind. Ed., 2004, 43, p. 5066-5069), Xu et al. (Org. Lett., Vol 6, No. 22, p. 4105-4107), Xu et al. (J. Org. Chem. 2005, 70, p. 8079-8087). Junge et al discloses a comparison of Ruthenium-based catalysts comprising phosphorous monodentate ligands for the enantioselective hydrogenation of beta-ketoesters. Junge et al tested several different types of monodentate phosphorous ligands: phosphines, phosphites, phosphonites and phosphoramidites. The best catalyst found by Junge et al was a phosphine ligand containing catalyst. The disadvantage of phosphines, however, is their lengthy synthesis.

Xu et al disclose the use of Ruthenium complexes comprising monodentate phosphorous ligands for the asymmetric ketone hydrogenation. The most effective catalyst found by Xu et al comprise a ligand comprising an aromatic group attached to the phosphor atom with either a methoxide or bromide on the ortho position of the aromatic ring. The disadvantage of phosphonites, however, is that the catalyst are prepared starting from RPCl2 compounds, of which there are only few commercially available.

However, there is still a need for an alternative method for the production of enantiomerically enriched chiral alcohols and amines in high yield and/or e.e. by asymmetric (transfer)hydrogenation of prochiral ketones, imines, oximes, oxime derivatives, hydrazones or hydrazone derivatives using a transition metal catalyst comprising an enantiomerically enriched chiral monodentate ligand.

The present invention relates to a method for the production of enantiomerically enriched chiral alcohols by asymmetric hydrogenation or asymmetric transfer hydrogenation of prochiral ketones and the production of enantioenriched chiral amines by asymmetric hydrogenation of imines, oximes, oxime derivatives, hydrazones or hydrazone derivatives using a transition metal catalysts, characterized in that as a catalyst is used a transition metal complex of the general formula [I]

MₐL¹_{b}L²_{c}(N)_{d}Xₑ [I]

wherein
a, b, c, d and e are integers; a, b and d can have a value of 1-6; c and e can have a value of 0-6;
M is transition metal selected from the group consisting of Ru, Rh and Ir;
L¹ is an enantiomerically enriched chiral monodentate phosphor-containing ligand of the general formula [II]

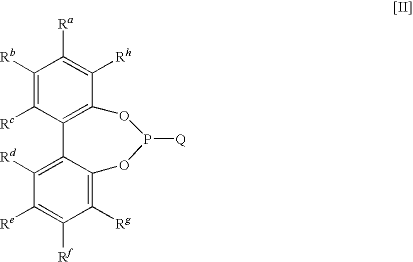

in which formula II, at least one of the C-atoms that form part of the ring at the positions to which the substituents R^{a}, R^{b}, R^{c}, R^{d}, R^{e}, R^{f}, R^{g} and R^{h} are attached, may be replaced by a heteroatom or a heteroatom containing group, such as —NH, O or S, or of the general formula [III]

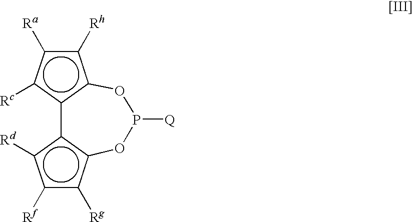

in which formula [III] at least one of the C-atoms that form part of the ring at the positions to which the substituents R^{a}, R^{c}, R^{d}, R^{f}, R^{g} and R^{h} respectively are attached, may be replaced by a heteroatom or a heteroatom containing group, such as —NH, O or S, and
wherein in the ligand of formula [III] at least one C-atom and at most three C-atoms not being one of the C-atoms connecting the two rings or those carrying the oxygen substituent that is part of the phosphoramidite functionality have been replaced by an O or S atom or an NR^{j} group or a combination thereof, and wherein R^{j} is H, an optionally substituted alkyl group optionally comprising one or more hetero atoms, or an optionally substituted aryl group, optionally comprising one or more hetero atoms R^{a}, R^{b}, R^{c}, R^{d}, R^{e}, R^{f}, R^{g}, R^{h} represent each independently a halide, an alkyl group which may be a straight chain alkyl group or which may be branched, and which alkyl group optionally comprises one or more hetero atoms and which alkyl group optionally is substituted, an aryl group which aryl group may optionally comprises one or more hetero atoms and which aryl group optionally is substituted, or each two adjacent R groups can together represent a ring structure, which ring structure may optionally contain one or more heteroatoms and which ring structure may also be substituted R^{a}, R^{b}, R^{e}, and R^{f} each independently also may be a hydrogen atom;
Q represents NRⁱR^{j} or OR^{k};
Rⁱ, R^{j} and R^{k} may be H, an alkyl group which may be a straight chain alkyl group or which may be branched, and which alkyl group optionally comprises one or more hetero atoms and which alkyl group optionally is substituted, an aryl group which aryl group may optionally comprises one or more hetero atoms and which aryl group optionally is substituted, or Rⁱ and R^{j} can together represent a ring structure, which ring structure may optionally containing one or more heteroatoms and which ring structure may also be substituted,
with the proviso that not both Rⁱ and R^{j} are hydrogen,
L² is any monodentate or bidentate neutral or monoanionic ligand, which may be chiral;
N is a compound containing at least one primary or secondary amine group.
X is an anion;

The term “enantiomerically enriched compound” means that one of the enantiomers of the compound is present in excess in comparison with the other enantiomer. This excess will hereinafter be referred to as “enantiomeric excess” or e.e. The e.e. may be determined for example by chiral GLC or HPLC analysis. The enantiomeric excess e.e. is equal to the difference between the amounts of enantiomers divided by the sum of the amounts of the enantiomers, which quotient can be expressed as a percentage after multiplication by 100.

With the term “ligand” is meant a group capable of binding with a transition metal, preferably by donating electron density to a transition metal atom.

With the term “monodentate” ligand is meant a ligand comprising one coordinating atom or group binding to the transition metal atom. With the term “bidentate” ligand is meant a ligand comprising two coordinating atoms or groups or a combination of a coordinating atom and coordinating group binding to the transition metal atom.

An example of a coordinating group is cyclopentadienyl, however, there are many others possible and known to a person skilled in the art.

Suitable ketones to be used in the method according to the invention are compounds according to formula [IV]:

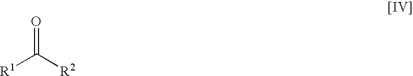

wherein R¹ and R² are not the same and represent each independently an alkyl group which may be a straight chain alkyl group or which may be branched, and which alkyl group optionally comprises one or more hetero atoms and which alkyl group optionally is substituted, an aryl group which aryl group optionally comprises one or more hetero atoms and which aryl group optionally is substituted, an alkenyl group or alkynyl group which alkenyl group or alkynyl group may be a straight chain alkenyl or alkynyl group or which may be branched, and which alkenyl or alkynyl group optionally comprises one or more hetero atoms and which alkenyl or alkynyl group optionally is substituted, or R¹ and R² can together represent a ring structure, which ring structure may optionally contain one or more heteroatoms and which ring structure may also be substituted.

Suitable substituents are for example halides, alkoxy, aryloxy, esters, amines, aromatic groups, alkyl groups. It will be clear to a person skilled in the art that the substituents themselves may also be substituted and may comprise hetero atoms.

Typical hetero atoms that may be present are N, O, S and P. Preferred substrates are ketones according to formula [IV] comprising a primary, secondary or tertiary amine group. The number of atoms in R¹ and R² may vary. Typically, R¹ and R² each comprise not more than 30 carbon atoms. Usually they each comprise between 1 and 20 C-atoms.

Suitable ketones to be used in the invention are for example acetophenone, 1-acetonaphthone, 2-acetonaphthone, 3-quinuclidinone, 2-methoxycyclohexanone, 1-phenyl-2-butanone, benzyl-isopropyl ketone, benzyl acetone, cyclohexyl methyl ketone, t-butylmethyl ketone, t-butylphenyl ketone, isopropyl phenyl ketone, ethyl-(2-methylethyl)-ketone, o-, m- or p-methoxyacetophenone, o-, m- or p-(fluoro, chloro,) acetophenone, o-, m- or p-cyanoacetophenone, o-, m- and/or p-trifluoromethyl-acetophenone, o-, m- or p-nitroacetophenone, 3,5-bis-trifluoromethyl-acetophenone, 2-acetylfluorene, acetylferrocene, 2-acetylthiophene, 3-acetylthiophene, 2-acetylpyrrole, 3-acetylpyrrole, 2-acetylfuran, 3-acetylfuran, 1-indanone, 2-hydroxy-1-indanone, 1-tetralone, p-methoxyphenyl-p′-cyanophenylbenzophenone, cyclopropyl-(4-methoxyphenyl)-ketone, 2-acetylpyridine, 3-acetylpyridine, 4-acetylpyridine, acetylpyrazine, alpha-haloketones, for example alpha-chloroacetophenone; alpha-keto acids, for example pyruvic acid, phenylglyoxylic acid, 4-phenyl-2-oxo-butyric acid, 3-oxo, 4,4-dimethyl-butyrolactone and esters and salts thereof; beta keto acids for example acetyl acetic acid, 4-phenylacetyl acetic acid, and esters and salts thereof; diketones, for example biacetyl, benzil, acetylacetone; hydroxyketones, for example hydroxyacetone, benzoin and 1-phenyl-1hydroxyacetone.

Other prochiral compounds that can be asymmetrically (transfer) hydrogenated according to the present invention are prochiral imines having the general formula [v]

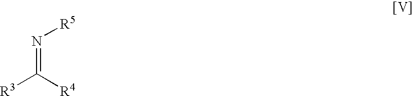

where R³ and R^{4,} are not the same and where R³, R⁵ and R⁵ each independently from one another represent an alkyl group which may be a straight chain alkyl group or which may be branched, and which alkyl group optionally comprises one or more hetero atoms and which alkyl group optionally is substituted, an aryl group which aryl group optionally comprises one or more hetero atoms and which aryl group optionally is substituted, an alkenyl group or alkynyl group which alkenyl group or alkynyl group may be a straight chain alkenyl or alkynyl group or which may be branched, and which alkenyl or alkynyl group optionally comprises one or more hetero atoms and which alkenyl or alkynyl group optionally is substituted, or R³ and R^{4,} can together represent a ring structure, which ring structure may optionally contain one or more heteroatoms and which ring structure may also be substituted.

Suitable substituents are for example halides, alkoxy, aryloxy, esters, amines, aromatic groups, alkyl groups. It will be clear to a person skilled in the art that the substituents themselves may also be substituted and may comprise hetero atoms. Typical hetero atoms that may be present are N, O, S and P. Preferred substrates are ketones according to formula [V] comprising a primary, secondary or tertiary amine group. The number of atoms in R³, R⁴ and R⁵ may vary. Typically, R³, R^{4,} and R⁵ each comprise not more than 30 carbon atoms. Usually they each comprise between 1 and 20 C-atoms.

R⁵ furthermore may be a group that can be split off, for example a dialkylsulfamoyl, phosphinyl, sulphonyl or benzyl group. Examples of imines are those prepared from the ketones described above and an alkyl amine or aryl amine or an amino acid derivative, for example an amino acid amide, an amino acid ester, a peptide or a polypeptide. Examples of a suitable alkyl amine or aryl amine are a benzyl amine, for example benzyl amine, or an o-, m- or p-substituted benzyl amine, an α-alkyl benzyl amine, a naphthyl amine, for example naphthyl amine, a 1,2,3,4,5,6,7 or 8-substituted naphthyl amine, a 1-(1-naphthyl)alkyl amine or a 1-(2-naphthyl)alkyl amine or a benzhydryl amine. Examples of suitable imines are N-(2-ethyl-6-methylphenyl)-1-methoxy-acetonimine, 5,6-difluoro-2-methyl-1,4-benzoxazine, 2-cyano-1-pyrroline, 2-ethyoxycarbonyl-1-pyrroline, 2-phenyl-1-pyrroline, 2-phenyl-3,4,5,6-tetrahydropyridine, 3,4-dihydro-6,7-dimethoxy-1-methyl-isoquinoline, 1-(p-methoxybenzyl)-3,4,5,6,7,8-hexahydroisoquinoline, N-diphenylphosphinyl 2-naphtophenone imine or N-tosyl-tetralone imine, (N,N′-dimethylsulfamoyl)-acetophenone imine.

The substrate may also be an oxime, an oxime derivative or a hydrazone or a hydrazone derivative according to formula (VI)

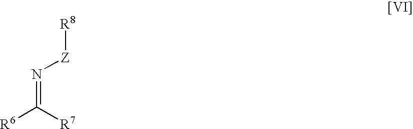

where —Z contains a heteroatom and represents NH, NR or O, for instance, with R representing an alkyl, aryl, aralkyl, alkenyl or alkynyl group, each with 1-20 C atoms.

R⁶ and R⁷ are not the same and R⁶, R⁷ and R⁸ each independently from one another represent an alkyl group which may be a straight chain alkyl group or which may be branched, and which alkyl group optionally comprises one or more hetero atoms and which alkyl group optionally is substituted, an aryl group which aryl group optionally comprises one or more hetero atoms and which aryl group optionally is substituted, an alkenyl group or alkynyl group which alkenyl group or alkynyl group may be a straight chain alkenyl or alkynyl group or which may be branched, and which alkenyl or alkynyl group optionally comprises one or more hetero atoms and which alkenyl or alkynyl group optionally is substituted, or R⁶ and R⁷ can together represent a ring structure, which ring structure may optionally contain one or more heteroatoms and which ring structure may also be substituted or the form a ring with R⁸ and the atoms to which they are bound, which ring may also contain one or more heteroatoms and may be substituted.

Suitable substituents are for example halides, alkoxy, aryloxy, esters, amines, aromatic groups, alkyl groups. It will be clear to a person skilled in the art that the substituents themselves may also be substituted and may comprise hetero atoms.

Typical hetero atoms that may be present are N, O, S and P. Preferred substrates according to formula [VI] comprising a primary, secondary or tertiary amine group. The number of atoms in R⁶, R⁷ and R⁸ may vary. Typically, R⁶, R⁷ and R⁸ each comprise not more than 30 carbon atoms. Usually they each comprise between 1 and 20 C-atoms.

In the case of an oxime or oxime ether, R⁸ is H or an alkyl, aryl, aralkyl, alkenyl, alkynyl, acyl, aryl phosphonyl, alkyl phosphonyl, aryl sulphonyl or alkyl sulfonyl group with 1-20 C-atoms, which groups may also contain one or more heteroatoms and may be substituted; and in the case of a hydrazone it is H, an alkyl, aryl, alkenyl, alkynyl, acyl, aryl phosphonyl, alkyl phosphonyl, aryl sulphonyl or alkyl sulfonyl group with 1-20 C-atoms, which groups may also contain one or more heteroatoms and may be substituted;

The method according to the invention is preferably carried out using substrates, i.e. ketones, imines, oxime, oxime-derivatives, hydrazone or hydrazone derivatives, wherein an aromatic group is present next to the functional group characterizing the substrate. For example, R⁶ or R⁷ is an aromatic group, or R³ or R⁴ is an aromatic group, or R¹⁵ or R¹⁶ is an aromatic group.

The metal M to be used in the catalyst used in the method according to the invention may be Ru, Rh or Ir. Ru is preferred.

Examples of suitable ligands L¹ according to the invention are

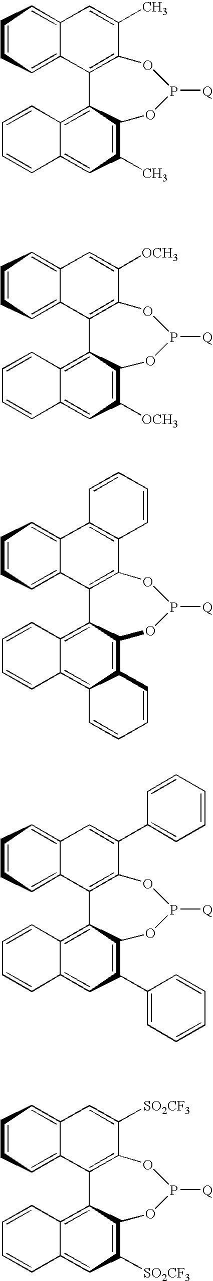

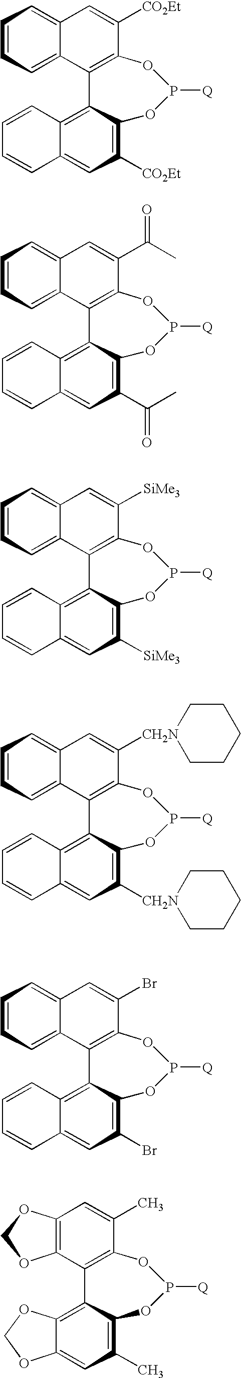

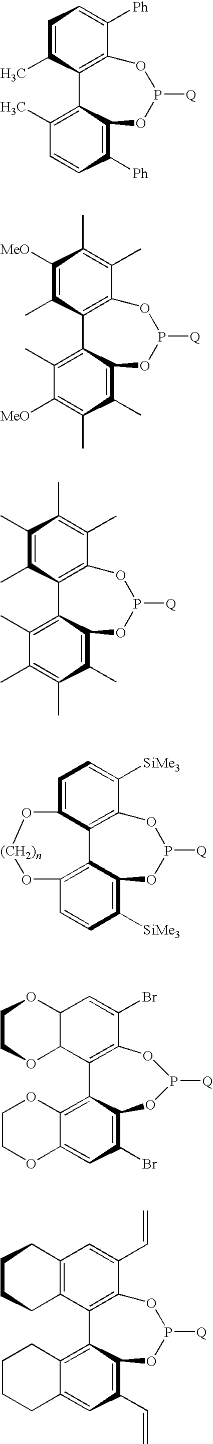

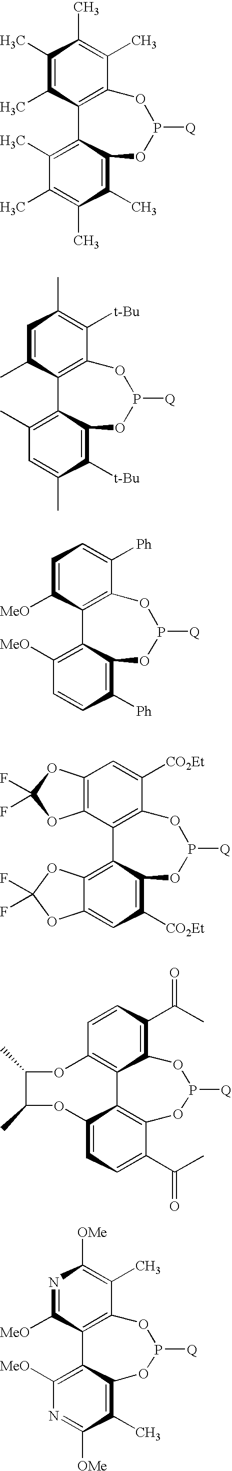

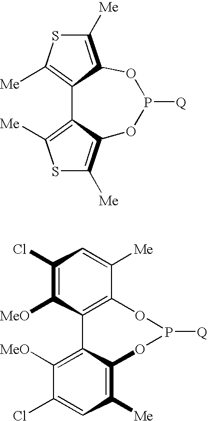

In the above structures Q=NRⁱR^{j} or OR^{k}. Suitable examples of Rⁱ, R^{j} and R^{k} are H, Me, Et, n-Pr, i-Pr, n-Bu, Ph, o-anisyl, p-tolyl, benzyl, 1-naphthyl, 2-naphthyl, 2-pyridyl, 3-pyridyl, (R) and (S)-alpha-methylbenzyl, 2-furyl, 3-furyl, 2-thiophenyl, 3-thiophenyl. Rⁱ and R^{j} may together with the nitrogen atom form a ring structure such as a pyrrolidine a piperidine a morpholine or a pyrrole structure. These rings may be fused to other rings or they may optionally be substituted. Rⁱ and R^{j} may be the same or different. They may not be both H.

It will be understood that where one enantiomer is represented, the other enantiomer is similarly applicable.

Such ligands with formula (I) can simply be prepared as described for example in Houben-Weyl Methoden der Organischen Chemie Band XII/2. Organische phosphorverbindungen. G. Thieme Verlag, Stuttgart, 1964, Teil 2 (4 th ed.), pp. 99-105. A preferred preparation method is based on the reaction of a compound of formula (VII)

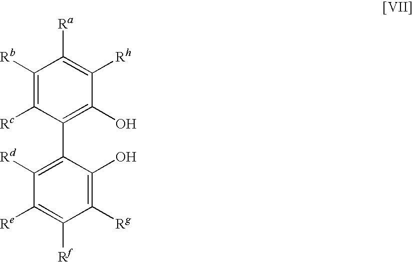

(wherein R^{a}, R^{b}, R^{c}, R^{d}, R^{e}, R^{f}, R^{g}, R^{h} are as define above)
with P(NMe₂)₃ or P(NEt₂)₃ (Me=methyl, Et=ethyl), with subsequent reaction with Q (RⁱR^{j} NH or R^{k} OH) preferably in a solvent having a boiling point >80° C., for example toluene. Examples of suitable catalysts for the latter reaction are ammonium chloride, tetrazole or benzimidazoliumtriflate. Examples of compounds of formula (VII) are 3,3′-disubstituted chiral bisnaphtols for example 3,3′-dimethyl-bis-1,1′-naphth-2,2′-ol, and chiral bisphenols for example 3,3′-bis-t-butyl-4,4′,5,5′-tetramethyl-bis-1,1′-phen-2,2′-ol,

A second preferred preparation is based on the reaction of a compound of formula (IX) with PCl₃, with subsequent reaction with Q, preferably in the presence of a base, for example Et₃ N, and in the presence of a solvent, for example toluene. Examples of a compound of formula (VII) are in principle the same as mentioned above in relation to the first preferred preparation.

A third preferred preparation starts with the reaction between Q and PCl₃, optionally in the presence of a base followed by reaction with the compound of structure (VII), preferably in the presence of a base. This method is particularly suited in case the compound Q is very bulky.

Ligands L¹ according to formula (III) may be prepared in analogues manner to the methods described above for ligands L¹ according to formula (II)

Preferably, L¹ is used with an e.e. >51%, more preferably with an e.e. >90%, most preferably, L¹ is used with an e.e >99%.

The number of atoms in each R may vary. If R^{a}, R^{b}, R^{c}, R^{d}, R^{e}, R^{f}, R^{g}, R^{h} comprise any C-atoms, they typically each comprise not more than 30 carbon atoms. Usually they each comprise between 1 and 20 C-atoms. Any substituents that may be present on R^{a}, R^{b}, R^{c}, R^{d}, R^{e}, R^{f}, R^{g}, R^{h} preferably comprise between 1-4 C-atoms.

The number of atoms in Rⁱ, R^{j} and R^{k} may vary. If Rⁱ R^{j} and R^{k} comprise any C-atoms, they typically each comprise not more than 30 carbon atoms. Usually they each comprise between 1 and 20 C-atoms. Any substituents that may be present on Rⁱ, R^{j} and R^{k} preferably comprise between 1-4 C-atoms. If Rⁱ and R^{j} form a ring together, that ring typically comprises not more than 20 C-atoms.

In a preferred embodiment, L¹ is a ligand synthesized starting from a 3,3′-substituted bi(2-naphtol) (BINOL) compound.

An exemplary ligand is (R)— or (S)-1-(2,6-Dimethyl-3,5-dioxa-4-phospha-cyclohepta[2,1-a;3,4-a′]dinaphthalen-4-yl)-piperidine ((R)— or (S) dimethyl PipPhos).

L² is any monodentate or bidentate neutral or monoanionic ligand, which may be chiral;

Examples of suitable ligands are ligands comprising P, N, S, or combinations thereof, as an electron density donating atom, or comprising carbon-based ligands in which a group of atoms rather than one atom donates electron density, or comprising combination of ligands with P, N, S or carbon-based ligands. Examples of suitable ligands L² are mono en bidentate phosphines, they may be tri-arylphosphines such as tri-phenylphoshine, tri-o-tolylphosphine, BINAP, Josiphos, tri-alkylphosphines such as trimethylphosphine, tri-butylphosphine, tri-cyclohexylphosphine, mixed phosphines such as methyldiphenylphosphine or Duphos, triarylphosphites, such as triphenylphosphite or 4,8-di-tert-butyl-6-(2-tert-butyl-phenoxy)-5,7-dioxa-6-phospha-dibenzo[a,c]cycloheptene, or tri-o-tBu-phenylphosphite, phosphonites such as (PhO)₂PPh, phosphonites such as P2₂POPh, pyridines, such as pyridine, 2-picoline, 3,5-lutidine, quinoline or isoquinoline, CO, cyclopentadienyl, pentamethyl-cyclopentadienyl, eta-6 benzene, eta-6 cumene; coordinated solvents such as THF or CH₃CN.

In a preferred embodiment, the method according to the invention the transition metal complex is used with a ligand L², thus, with c=0.

N is a compound containing at least one primary or secondary amine group. N may be chiral or non-chiral. For example, the amine containing compound N may be a monoamine such as for example benzylamine, pentylamine or 2-aminopyridine, it may be a diamine such as for example 1, 2 ethylenediamine, 1,2-phenylenediamine, (R,R)— or (S,S)-1,2-diphenyl-1,2-ethylendiamine (DPEN), or (R,R)— or (S,S)-1,2-cyclohexanediamine (DACH); it may be an aminoalcohol, such as for example (R,R)-1-amino-2-indanol or 2-aminophenol, it may be an aminothioether, such as for example 2-methylthioaniline or 1-amino-1-phenyl-2-methylthiopropane.

In a preferred embodiment the compound N in formula (II) is an optionally substituted vicinal ethylenediamine of the general formula [IV]

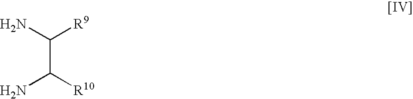

wherein R⁹ and R¹⁰ each independently may represent optionally substituted alkyl, aryl, alkyl-aryl or aryl-alkyl, or R⁹ and R¹⁰ together may form an optionally substituted ring structure, optionally containing heteroatoms.

Most preferred compounds N are (R,R)-DPEN, (S,S)-DPEN, (R,R)-DACH and (S,S)-DACH.

X is an anion, typically a monovalent or bivalent anion. Examples of suitable anions for the purpose of the present invention are CI, Br, I, OAc, BF₄, PF₆, ClO₄, p-toluene sulphonate, benzene phosphonate, tetra-pentafluorophenylborate. Halides are preferred anions, in particular bromide and chloride. In case the catalyst is anionic it may contain an additional cation. Examples of suitable cations are for example alkali metals, for example Li, Na or K, alkaline earth metals such as for example Mg or Ca, or ammonium, or alkyl-substituted ammonium.

The catalyst may contain a hydride, which is usually introduced by reduction of one or more of the halide ions that are part of the complex. For instance if MₐL¹_{b}L²_{c}N_{d}Xₑ is treated with a reductant such as hydrogen or a hydride reagent, such as sodium borohydride new complexes may form such as MₐL¹_{b}L²_{c}N_{d}Xₑ₋₁H or MₐL¹_{b}L²_{c}N_{d}Xₑ₋₂H₂. All three types of complexes are considered to be catalysts of the invention.

The catalyst suitable for use in the method according to the invention represented by the formula (I) may be neutral, anionic or cationic.

The catalyst suitable for use in the method according to the invention may consist of a preformed complex having the formula I These complexes can be prepared by reacting the ligand L¹ and the ligand L² either together as a mixture or one after the other with a suitable catalyst precursor. Thereafter the product formed from this reaction is again reacted with the amino compound N. If necessary the product of this reaction is purified. The complex thus obtained may be used ads the catalyst of the invention. Alternatively it may be desirable to change the counterion X of this complex for instance by reacting the complex with HX or by anion exchange following established methods. In exceptional cases, it may be possible to form the catalyst in situ by adding the ligands L¹ and optionally L² and N together to a solution of a catalyst precursor. The catalyst precursor contains at least the metal M. The precursor may contain ligands that are easily displaced by the ligands L¹ and or L² and or N or it may contain a ligand that is easily removed by hydrogenation. In most cases the precursor already contains an anion, which may already be the same as X. It is also possible that the catalyst precursor already contains ligand L² although the ratio between M and L² may be different from that in the final catalyst I. The optimum ratio of ligands L₁, ligand L² and amine N to the metal in the catalyst may differ per ligand and per amine and per metal and can readily be determined by means of experiments. In a preferred embodiment, the catalyst is activated by a base. Suitable bases are for example nitrogen bases for instance triethylamine, DBU, and substituted or non-substituted pyridines and mineral bases for example KOtBu or Cs₂CO₃.

If desired, the catalyst can be activated by means of hydrogenation or reduction prior to the addition of the substrate. In most cases, this will not be necessary.

Examples of suitable catalyst precursors are RuCl₃, RuCl₃.nH₂O, [RuCl₂(η⁶-benzene)]₂, [RuCl₂(η⁶-cymene)]₂, [RuCl₂(η⁶-mesitylene)]₂, [RuCl₂(η⁶-hexamethylbenzene)]₂, [RuCl₂(η⁶-1,2,3,4-tetramethylbenzene)]₂, [RuBr₂(η⁶-benzene)]₂, [RuI₂(η⁶-benzene)]₂, trans-[RuCl₂(DMSO)₄], RuCl₂(PPh₃)₃, Ru(COD)(COT), (in which COD=1,5-cyclooctadiene and COT=1,3,5-cyclooctatriene) Ru(COD)(methylallyl)₂, IrCl₃, [Ir(COD)Cl]₂, [Ir(CO)₂Cl]ₙ, [IrCl(CO)₃]ₙ, [Ir(CP*)Cl₂]₂, Ir(Acac)(COD), [Ir(NBD)Cl]₂, [Ir(COD)(C₆H₆)]⁺BF₄⁻, [(CF₃C(O)CHC(O)CF₃)Ir(COE)₂], (in which COE is cyclooctene) [Ir(CH₃CN)₄]⁺BF₄⁻, IrCl(CO)(PPh₃)₂, [Rh(C₆H₁₀)Cl]₂(in which C₆H₁₀=hexa-1,5-diene), [Rh(COD)Cl]₂, [Rh(Cp)(CO)₂], [Rh(norbornadiene)₂]BF₄, [Rh(Cp*)Cl₂]₂(in which Cp* is pentamethylcyclopentadienyl).

Examples of fully prepared catalysts of the invention are: Ru(L¹)₂(DACH)Cl₂ in which L¹ is 1-(2,6-Dimethyl-3,5-dioxa-4-phospha-cyclohepta[2,1-a;3,4-a′]dinaphthalen-4-yl)-piperidine, Ir(COD)(L¹)Cl in which L¹=(R)—O,O′-(4,4′,5,5′-tetramethyl-3,3′-bis-tert-butyl-1,1′-biphenyl-2,2′-diyl),N,N′-dimethyl-phosphoramidite or Rh(Cp*)(L¹)(S,S-DPEN) in which L¹=(S)—O,O′-(3,3′-diphenyl-1,1′-dinaphthyl-2,2′-diyl)-tert-butylphosphite.

The use of the catalysts in the method according to the invention takes place in the presence of one or more hydrogen donors, which in the context of this invention are understood to be compounds that can in some way transfer hydrogen to the substrate. All known hydrogen donors for hydrogenation or transferhydrogenation reactions may be used in the method according to the invention. Suitable hydrogen donors for example are H₂, aliphatic or benzylic alcohols with 1-10 C-atoms, in particular secondary alcohols with 1-10 C-atoms, for example isopropanol or cyclohexanol, or unsaturated hydrocarbons with 5-10 C-atoms, for example 1,4 dihydrobenzene or hydroquinone, reducing sugars, for example glucose or derivates of formic acid, for example salts of formic acid, such as for example ammonium formate. It is also possible to use for example an azeotropic mixture of formic acid and triethylamine. H₂ is preferred for carrying out hydrogenation reactions according to the invention, and isopropanol is preferred for carrying out transferhydrogenation reactions.

The molar ratio of substrate to hydrogen donor preferably lies between 1:1 and 1:100. The hydrogen pressure may vary within wide limits and is preferably chosen to be as high as possible when a fast reaction or the lowest possible amount of catalyst is desired. The hydrogen pressure for example lies between 0.05 and 20 MPa, preferably between 0.1 and 10 MPa, in particular between 0.15 and 8 MPa.

In the asymmetric hydrogenation use is preferably made of a molar ratio of metal present in the transition metal compound to substrate of between 1:10 and 1:1,000,000, in particular between 1:50 and 1:100,000.

The temperature at which the asymmetric (transfer) hydrogenation is carried out is generally a compromise between reaction velocity and enantioselectivity, and preferably lies at or above −20° C., more preferably at or above −10° C. and most preferably at or above 0° C. The temperature at which the asymmetric (transfer) hydrogenation is carried out preferably lies at or below 120° C., more preferably at or below 80° C., and most preferably at or below 60° C. The asymmetric (transfer) hydrogenation is preferably carried out with O₂ being excluded. Preferably the substrates and solvents do not contain any O₂, peroxides or other oxidizing substances.

As solvent use can be made of: alcohols, esters, amides, ethers, ketones, aromatic hydrocarbons, halogenated hydrocarbons. Preferably use is made of ethyl acetate, 2-propanol, acetone, tetrahydrofuran (THF), dichloroethane or toluene. It is also possible to carry out the asymmetric (transfer) hydrogenation in ionic liquids as described in T. Welton, Chem. Rev., 99, 2071-2083 (1999), so that isolation of the product is simplified. If necessary the solubility of the ligand in the ionic liquid can be increased by providing the ligand with polar groups such as carboxylate salts. If the substrate is a liquid, the hydrogenation can also very suitably be carried out without a solvent. If the substrate and/or the product hardly dissolves in the solvent the asymmetric (transfer) hydrogenation can also be performed as a slurry. If the product forms a slurry, its isolation is very much simplified.

Preferably the (transfer) hydrogenation reaction is carried out without preceding prehydrogenation. However, it is also possible to activate the catalyst for the asymmetric (transfer) hydrogenation prior to the addition of the substrate by hydrogenation with hydrogen or by treatment with a reducing agent, for example NaBH₄. The (transfer) hydrogenation reaction will sometimes also be accelerated by adding a base, an acid, a halide, or an N-hydroxyimide prior to or during the hydrogenation. Suitable acids are for example HBr, trifluoroacetic acid. Suitable halides are for example alkali halides or tetraalkylamonium halides e.g. LiI, LiBr, LiCl, NaI, tetrabutylammonium iodide. A suitable N-hydroxy-imide is for instance N-hydroxy-phtalic-imide.

Using the process according to the invention, enantiomerically enriched compounds may be obtained with an e.e. of 75% or higher, in particular >85%, more in particular >90%. Preferably an e.e. of >95% is obtained.

In this text, for aspects of the method according to the invention preferred ranges, compositions or embodiments have been described. The invention explicitly covers the combination of each preferred feature or each embodiment individually with the method according to claim 1 as well as all possible combinations of one or more preferred features or embodiments with the method according to claim 1, and also any possible combination of preferred features with catalyst complexes MₐL¹_{b}L²_{c}N_{d}Xₑ, and its hydride forms MₐL¹_{b}L²_{c}N_{d}Xₑ₋₁H or MₐL¹_{b}L²_{c}N_{d}Xₑ₋₂.

The invention will be elucidated with reference to the following examples, without however being restricted by these:

### EXAMPLE 1

### Synthesis of (S)-1-(2,6-Dimethyl-3,5-dioxa-4-phospha-cyclohepta[2,1-a;3,4-a′]dinaphthalen-4-yl)-piperidine ((S)-3-3′-DimethylPipPhos)

2 gram (6,4 mmol) of 3-3′-dimethyl-bis-β-naphtol was dissolved in 10 ml of PCl₃. The solution was refluxed under N₂ for 16 hours. Hereafter, the excess of PCl₃ was distilled off in vacuo. The residual solid was subjected to azeotropic distillation with toluene (2×10 ml), resulting in the crude chlorophosphite.

The chlorophosphite was redissolved in toluene (10 ml) under nitrogen. To the solution 2 ml Et₃N (14,1 mmol, 2,2 equiv) was added. Next 0.70 ml of piperidine (7 mmol, 1.1 equiv) was added in small portions. After two hours of stirring 10 ml MTBE was added. The resulting suspension was filtered over Celite and the filtrate was purified over silica using pentane/ethyl acetate (9:1) as eluent.

### EXAMPLE 2

### Synthesis of a Catalyst Comprising Ru, 3,3′-dimethyl-PipPhos and ((S,S)-DPEN)]

A Schlenk flask was flame-dried and 62 mg [RuCl₂(cymene)]₂ (0.1 mmol) and 160 mg of (S)-3,3′-dimethyl-PipPhos (0.4 mmol) were added. The Schlenk tube was degassed by three cycles of vacuum/N₂ and then kept under N₂ and the solids were dissolved in 5 ml of dry DMF. This mixture was heated for 3 hours at 90° C. After three hours the mixture was let to cool off to room temperature. When room temperature was reached 43 mg of (S,S)-DPEN (0.2 mmol) was added. This solution was stirred over night. After over night stirring DMF was evaporated under reduced pressure. The resulting solid mass was subjected to azeotropic distillation with toluene (2×5 ml) and washed twice with 5 ml hexane. The obtained solid was used in hydrogenation reactions without further purification.

### EXAMPLE 3

### Synthesis of a Catalyst Comprising Ru, 3,3′-dimethyl-PipPhos and ((R,R)-DACH)]

The experiment described in Example 3 was repeated with the difference that in this case (R,R)-diaminocyclohexane (DACH) was used.

### EXAMPLE 4

### Synthesis of a Catalyst Comprising Ru, ((R)-3,3′-dimethyl MonoPhos) and (R)-DPEN)]

The experiment, described in example 2 was repeated with the difference that instead of (S)-3,3′-dimethyl-PipPhos, (R)-3,3′-dimethyl-monoPhos was used and (R)-DPEN instead of (S)-DPEN.

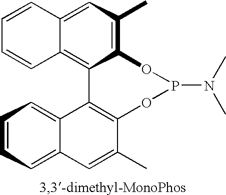

### EXAMPLES 5-15

### Procedure for the Hydrogenation of Aromatic Ketones

These experiments were performed in small autoclaves that can be pressurized to 25 bar. To a glass liner for an autoclave 2 mmol of substrate is added. To the substrate is added 0.1 mol % of preformed catalyst of example 3 and 10 μl of a 1 M solution of KOtBu in iPrOH. To this 3.7 ml iPrOH is added. The liner is put into one of the parallel autoclaves and, while stirring, 25 bars of hydrogen pressure is applied. After 24 hours the autoclave is carefully vented and the glass liner is taken out. From the reaction mixture a sample is taken and filtered over a silica plug to prepare a GC sample. The sample containing the product alcohol is analyzed by chiral GC.

According to this procedure the following substrates were reacted:

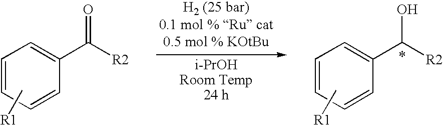

| | | | Enantiomeric |
|---|---|---|---|
| Example | Substrate | Yield | excess (ee) |
| 5 | Acetophenone | 100% | 97% |
| 6 | 2-Methyl-acetophenone | 100% | 97% |
| 7 | 2-Methoxy-acetophenone | 100% | 96% |
| 8 | 4-methoxyacetophenone | 100% | 97% |
| 9 | 4-Chloro-acetophenone | 100% | 95% |
| 10 | 3-Bromo-acetophenone | 100% | 65% |
| 11 | 3-Chloro-acetophenone | 100% | 83% |
| 12 | 3,5-Bis(trifuoromethyl)- | 100% | 95% |
| | acetophenone | | |
| 13 | 1-Acetonaphthone | 100% | 93% |
| 14 | 2-Acetonaphtone | 100% | 94% |
| 15 | n-Propiophenone | 100% | 91% |

### EXPERIMENT 16

The experiment of Example 5 was repeated with the difference that the catalyst of Example 2 was used. The product alcohol was obtained in 100% yield and 97% ee.

### EXPERIMENT 17

The experiment of Example 5 was repeated with the difference that the catalyst of Example 4 was used. The product alcohol was obtained in 100% yield and 90% ee.

### COMPARATIVE EXAMPLE 18

The experiment of Example 5 was repeated with the difference that [Ru((R)-PipPhos)((R)-DACH)Cl₂] was used as catalyst. This is not a catalyst of the invention. The product alcohol was obtained in 100% yield and 55% ee.

### COMPARATIVE EXAMPLE 19

The experiment of Example 5 was repeated with the difference that [Ru((R)-PipPhos)((R)-DPEN)Cl₂] was used as catalyst. This is not a catalyst of the invention. The product alcohol was obtained in 100% yield and 52% ee.

Thus, experiments 18 and 19 show that it is necessary to have a substituent, other than hydrogen on the carbon atoms adjacent to the carbon atoms that are part of the cyclic phosphoramidite ring and that are attached to the oxygen atoms in the ring, to induce a high selectivity in the product.

### EXAMPLE 20

The experiment of Example 6 was repeated with the difference that a catalyst was used that was prepared in situ from [Ir(COD)Cl]₂, (R)-3,3′-dimethyl-PipPhos and (R)-DACH. The product alcohol was obtained in 86% yield and 67% ee.

1.-4. (canceled)

5. An asymmetric hydrogenation or asymmetric transfer hydrogenation method comprising subjecting a prochiral keton, a prochiral imine, an oxime, an oxime derivative, a hydrazone or a hydrazone derivative to asymmetric hydrogenation or asymmetric transfer hydrogenation conditions in the presence of a transition metal complex catalyst of the general formula [I]:
MaL¹bL²c(N)dXe [I]
wherein
a, b, c, d and e are integers, wherein a, b and d can have a value of 1-6, and c and e can have a value of 0-6;
M is transition metal selected from the group consisting of Ru, Rh and Ir;
L¹ is an enantiomerically enriched chiral monodentate phosphor-containing ligand of the general formula [II]: 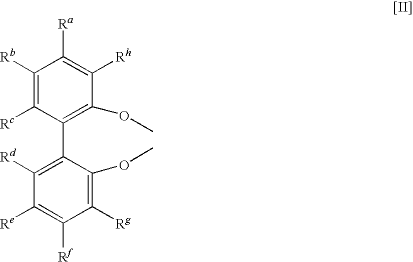
wherein in formula II, at least one of the C-atoms that forms part of the ring at the positions to which the substituents R^{a}, R^{b}, R^{c}, R^{d}, R^{e}, R^{f}, R^{g} and R^{h} are attached, may be replaced by a heteroatom or a heteroatom containing group, such as —NH, O or S, or of the general formula [III] 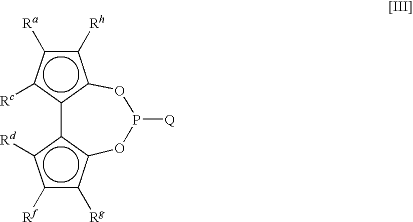
wherein in formula [III] at least one of the C-atoms that form part of the ring at the positions to which the substituents R^{a}, R^{c}, R^{d}, R^{f}, R^{g} and R^{h} respectively are attached, may be replaced by a heteroatom or a heteroatom containing group, such as —NH, O or S, and wherein in the ligand of formula [III] at least one C-atom and at most three C-atoms not being one of the C-atoms connecting the two rings or those carrying the oxygen substituent that is part of the phosphoramidite functionality have been replaced by an O or S atom or an NR^{j} group or a combination thereof, and wherein R^{j} is H, an optionally substituted alkyl group optionally comprising one or more hetero atoms, or an optionally substituted aryl group, optionally comprising one or more hetero atoms;
R^{a} R^{b}, R^{c}, R^{d}, R^{e}, R^{f}, R^{g}, R^{h} each independently represents a halide, an alkyl group which may be a straight chain alkyl group or which may be branched, and which alkyl group optionally comprises one or more hetero atoms and which alkyl group optionally is substituted, an aryl group which aryl group may optionally comprise one or more hetero atoms and which aryl group optionally is substituted, or each two adjacent R groups can together represent a ring structure, which ring structure may optionally contain one or more heteroatoms and which ring structure may also be substituted R^{a}, R^{b}, R^{e}, and R^{f} each independently also may be a hydrogen atom;
Q represents NRⁱR^{j} or OR^{k};
Rⁱ, R^{j} and R^{k} may be H, an alkyl group which may be a straight chain alkyl group or which may be branched, and which alkyl group optionally comprises one or more hetero atoms and which alkyl group optionally is substituted, an aryl group which aryl group may optionally comprise one or more hetero atoms and which aryl group optionally is substituted, or Rⁱ and R^{j} can together represent a ring structure, which ring structure may optionally contain one or more heteroatoms and which ring structure may also be substituted with the proviso that not both Rⁱ and R^{j} are hydrogen;
L² is any monodentate or bidentate neutral or monoanionic ligand, which may be chiral;
N is a compound containing at least one primary or secondary amine group; and
X is an anion.

6. A method according to claim 5, wherein N is an optionally substituted vicinal ethylenediamine of the general formula [IV]: 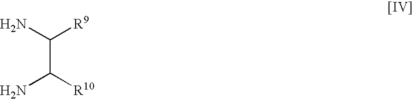
wherein R⁹ and R¹⁰ each independently may represent optionally substituted alkyl, aryl, alkyl-aryl or aryl-alkyl, or R⁹ and R¹⁰ together may form an optionally substituted ring structure, optionally containing heteroatoms.

7. Method according to claim 5, wherein L¹ is a ligand synthesized from a 3,3′-disubstituted 1,1′-bi(2-naphtol) starting compound.

8. Method according to claim 5, wherein L¹ is (S)— or (R)-1-(2,6-Dimethyl-3,5-dioxa-4-phospha-cyclohepta[2,1-a;3,4-a′]dinaphthalen-4-yl)piperidine.

9. An asymmetric hydrogenation or asymmetric transfer hydrogenation method comprising subjecting a prochiral keton, a prochiral imine, an oxime, an oxime derivative, a hydrazone or a hydrazone derivative to asymmetric hydrogenation or asymmetric transfer hydrogenation conditions in the presence of a transition metal complex catalyst of the general formula [I]:
MaL¹bL²c(N)dXe [I]
wherein
a, b, c, d and e are integers wherein a, b and d can have a value of 1-6, and c and e can have a value of 0-6;
M is transition metal selected from the group consisting of Ru, Rh and Ir;
L¹ is (S)— or (R)-1-(2,6-Dimethyl-3,5-dioxa-4-phospha-cyclohepta[2,1-a;3,4-a′]dinaphthalen-4-yl)-piperidine;
L² is any monodentate or bidentate neutral or monoanionic ligand, which may be chiral;
N is an optionally substituted vicinal ethylenediamine of the general formula [IV]: 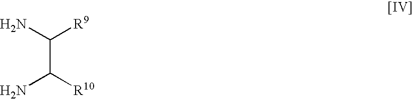
wherein R⁹ and R¹⁰ each independently may represent optionally substituted alkyl, aryl, alkyl-aryl or aryl-alkyl, or R⁹ and R¹⁰ together may form an optionally substituted ring structure, optionally containing heteroatoms; and
X is an anion.
